(19) United States
(12) Patent Application Publication
(10) Pub. No.: US 2026/0013883 A1
(43) Pub. Date: Jan. 15, 2026

(54) CUTTING GUIDE INCLUDING A BALL-JOINT FOR FACILITATING BONE RESECTION

(71) Applicants: Smith & Nephew, Inc., Memphis, TN (US); Smith & Nephew Orthopaedics AG, Zug (CH)

(72) Inventors: Ethan Fassezke, Pittsburgh, PA (US); Samuel C. Dumpe, Sewickley, PA (US); Mara C. Palmer, Pittsburgh, PA (US); James Kromka, Monroeville, PA (US)

(73) Assignees: Smith & Nephew, Inc., Memphis, TN (US); Smith & Nephew Orthopaedics AG, Zug (CH)

(21) Appl. No.: 19/244,154

(22) Filed: Jun. 20, 2025

Related U.S. Application Data
(60) Provisional application No. 63/668,965, filed on Jul. 9, 2024.

(51) Int. Cl. A61B 17/17; A61B 17/15

(52) U.S. Cl. CPC A61B 17/1778; A61B 17/151

(57) ABSTRACT

A cutting guide for resecting a portion of a patient's bone such as, for example, a humeral head of a patient's humerus. The cutting guide including a body component, a cutting slot component including a cutting slot configured to receive a cutting instrument for resecting the portion of the patient's bone, a ball-joint coupling or clamping mechanism for coupling the cutting slot component to the body component, and a quick release mechanism moveable between a first position and a second position. In the first position, the release mechanism applies a clamping or spring force on the ball-joint coupling or clamping mechanism to inhibit movement of the cutting slot component relative to the body component. In the second position, the release mechanism releases the clamping or spring force on the ball-joint coupling or clamping mechanism so that the cutting slot component can be moved (e.g., rotated) relative to the body component.

### CROSS-REFERENCE TO RELATED APPLICATIONS

This is a non-provisional of, and claims the benefit of the filing date of, U.S. provisional patent application No. 63/668,965, filed Jul. 9, 2024, entitled “Cutting Guide Including a Ball-Joint for Facilitating Bone Resection,” the entirety of which application is incorporated by reference herein.

### FIELD OF THE DISCLOSURE

The present disclosure relates generally to orthopedic devices, systems, and methods and more particularly to a cutting guide arranged and configured to resect a patient's bone. For example, the cutting guide may be used to resect the patient's humeral head during a total shoulder arthroplasty.

### BACKGROUND OF THE DISCLOSURE

Many surgical procedures require preparation of a bone surface to receive an implant. In the case of a shoulder procedure, such as a total shoulder arthroplasty, a shoulder implant may be used to replace the natural head of the patient's humerus. For example, a humeral implant may be inserted within the patient's humerus. The humeral implant may include an intramedullary stem component, which is implanted into the intramedullary canal of the patient's humerus and a hemi-spherically-shaped prosthetic head component or cup component, depending on the configuration of the surgical implant, which is secured to the stem component. In a total shoulder arthroplasty, the natural humeral head of the patient's humerus is resected. During resection of the humeral head, a planar cut is made to remove the uppermost portion of the patient's humerus (e.g., planar cut is made to remove portions of the humeral head). Resection of the humeral head is a critical aspect of a total shoulder arthroplasty as it defines the placement of the subsequently implanted humeral implant.

General speaking, to resect the patient's humeral head, a cut is made to expose the shoulder joint including the upper humeral shaft and the humeral head. Next, the humeral head may be dislocated and exposed. Finally, the humeral head may be resected. In resecting the humeral head, some surgeons prefer to identify the anatomic neck. During the procedure, surgeons may mark the anatomic neck, for example, mark the angle of the anatomic ridge along the anterior surface of the humerus to define the inclination of the planar cut. In addition, the surgeon may mark the angle of the anatomic ridge along, for example, the medial or lateral surface of the humerus to define the version of the planar cut. Thereafter, using a cutting instrument or saw and the markings, the humeral head may be resected using a free-hand technique. Alternatively, some surgeons prefer to utilize either an intramedullary or extramedullary cutting guide. Depending on the cutting guide being utilized, the cutting guide may be attached to the patient's humerus either via an intramedullary stem inserted into the intramedullary canal of the patient's humerus, or attached to the exterior surface of the humerus. In either event, the cutting guide includes a cutting slot to guide the cutting instrument or saw for resecting the patient's humeral head. General speaking, the position of the cutting slot can be adjusted, however doing so is typically cumbersome and time consuming. For example, current cutting guides are selectively adjusted and secured using a plurality of screws, one degree of freedom at a time. This increases time and also limits range of motion. In use, because range of motion is limited, initial placement of the cutting slot is much more critical/challenging.

It would be beneficial to provide a cutting guide that enables precise placement of the cutting slot and which is easier and faster to use (e.g., a cutting guide that can be positioned with ease and provide increased accuracy and/or flexibility). In addition, it would be beneficial if the cutting guide enabled tracking during a computer-assisted surgical procedure. It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An instrument or cutting guide for resecting portions of a patient's bone is provided. In some examples, the cutting guide includes a cutting slot arranged and configured to receive a cutting instrument or saw arranged and configured to resect (i.e., cut) a patient's bone. For example, the cutting guide may be used in a total shoulder arthroplasty (TSA) procedure to resect a humeral head of the patient's humerus. Alternatively, the cutting guide may be used in a hip procedure to resect a femoral head of the patient's femur or a knee procedure to resect portions of the patient's femur and/or tibia.

In some examples, the cutting guide is arranged and configured to be used in a computer-assisted surgical system to enable tracking of the cutting slot.

In some examples, the cutting guide includes a first or body component and a second or cutting slot component. The cutting slot component including a cutting slot arranged and configured to receive the cutting instrument or saw. The cutting slot component being coupled to the body component via a ball-joint coupling or clamping mechanism for selectively positioning and securing the position of the cutting slot component relative to the body component.

In addition, the cutting guide may include a push-button or release tab moveable between first and second positions. In the first position, the ball-joint coupling or clamping mechanism is locked so that movement of the cutting slot component relative to the body component is prevented, or at least inhibited. In the second position, the ball-joint coupling or clamping mechanism is released so that the cutting slot component can be moved (e.g., rotated) relative to the body component thereby enabling the surgeon to position the cutting slot in a desired position.

Thus arranged, the cutting guide uses a ball-joint to allow for the cutting slot in, for example, a total shoulder arthroplasty (TSA) procedure to be navigated and secured in place with a high level of adjustability and speed.

In some examples, a cutting guide for resecting a portion of a patient's bone is disclosed. The cutting guide including a body component, a cutting slot component including a cutting slot arranged and configured to receive a cutting instrument or saw for resecting the portion of the patient's bone, a ball-joint coupling mechanism for coupling the cutting slot component to the body component, the ball-joint coupling mechanism including a ball and a socket, and a release mechanism moveable between a first position and a second position, wherein, in the first position, the release mechanism applies a clamping or spring force on the ball-joint coupling mechanism to inhibit movement of the cutting slot component relative to the body component, and in the second position, the release mechanism releases the clamping or spring force on the ball-joint mechanism so that the cutting slot component can be rotated relative to the body component.

In any preceding or subsequent example, the release mechanism includes a push-button tab including first and second arms, wherein pressing the first and second arms towards each other causes the release mechanism to move from the first position to the second position.

In any preceding or subsequent example, pressing the first and second arms toward each other causes an opposite end of the first and second arms to move away from each other causing the clamping or spring force on the ball-joint coupling mechanism to be reduced.

In any preceding or subsequent example, the first and second arms are biased apart so that the release mechanism is biased toward the first position.

In any preceding or subsequent example, the cutting slot component further includes a locking screw wherein tightening the locking screw further secures a relative position of the cutting slot component relative to the body component.

In any preceding or subsequent example, the cutting slot component further includes an opening for receiving a bone pin for further securing the position of the cutting slot component relative to the body component.

In any preceding or subsequent example, the cutting device further includes a shaft, the ball being located at a first end of the shaft, the shaft including a plurality of splines at a second end thereof.

In any preceding or subsequent example, the body component includes an opening for receiving the shaft therein and a knob for engaging the plurality of splines formed on the shaft so that, in use, rotation of the knob translates the shaft, and hence the cutting slot component relative to the body component along a longitudinal axis of the shaft.

In any preceding or subsequent example, in the second position, the ball-joint coupling mechanism enables multiple rotational degree of freedoms to enable a surgeon to position the cutting slot component relative to the body component.

In any preceding or subsequent example, the ball-joint coupling mechanism enables simultaneous adjustment of an inclination angle and a version angle of a resection plane.

In any preceding or subsequent example, the body component is coupled to the patient's bone via a coupling mechanism.

In any preceding or subsequent example, the coupling mechanism includes a clamping mechanism arranged and configured to receive one or more bone pins.

In any preceding or subsequent example, the clamping mechanism includes a clamp plate and a threaded knob, in use, rotation of the threaded knob moves the clamp plate relative to the body component to secure the body component to the one or more bone pins.

In any preceding or subsequent example, the cutting guide further includes a plane visualizer arranged and configured to be at least partially inserted into the cutting slot, the plane visualizer including a plurality of fiducial markers to assist in locating and tracking a position of the cutting slot.

In any preceding or subsequent example, the cutting guide further includes a tracking array including a plurality of fiducial markers to assist in locating a position of the patient's bone.

In any preceding or subsequent example, an outer surface of the ball and an inner surface of the socket comprise a textured surface.

Examples of the present disclosure provide numerous advantages. For example, in accordance with one or more features of the present disclosure, the cutting guide incorporates a ball-joint coupling or clamping mechanism to enable a high level of adjustability and speed eliminating, or at least minimizing, the challenging series of steps associated with prior art devices, along with reducing the difficulty and inaccuracy, or permitted limited flexibility and adjustment of current cutting devices after initial placement. In addition, the cutting guide of the present disclosure is arranged and configured to be used in a computer-assisted surgical system to enable tracking of the cutting slot.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIGS. 1A-1E illustrate various perspective views of an example of a cutting guide in accordance with one or more features of the present disclosure, the cutting guide shown coupled to a patient's humerus; and

FIGS. 2A and 2B illustrate alternate perspective views of the cutting guide shown in FIGS. 1A-1E, the cutting guide being coupled to a plane visualizer and a tracking array in accordance with one or more features of the present disclosure.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of an instrument or cutting guide device, system, and corresponding method of use, will now be described more fully herein with reference to the accompanying drawings, in which one or more features of the cutting guide will be shown and described. It should be appreciated that the various features may be used independently of, or in combination, with each other. It will be appreciated that the cutting guide as disclosed herein may be embodied in many different forms and may selectively include one or more concepts, features, or functions described herein. As such, the cutting guide should not be construed as being limited to the specific examples set forth herein. Rather, these examples are provided so that this disclosure will convey certain features to those skilled in the art.

In accordance with one or more features of the present disclosure, the cutting guide includes a first or body component and a second or cutting component. The cutting component may include a slot. The cutting component being coupled to the body component via a ball-joint coupling or clamping mechanism (terms coupling or clamping used interchangeable herein without the intent to limit or distinguish) for selectively positioning and securing the position of the cutting component (e.g., cutting slot) relative to the body component. In addition, the cutting guide may include a quick release mechanism such as, for example, a push-button or release tab moveable between first and second positions. In the first position, the ball-joint coupling mechanism is locked so that movement of the cutting component relative to the body component is prevented, or at least inhibited. In the second position, the ball-joint coupling mechanism is released so that the cutting component can be moved (e.g., rotated) relative to the body component thereby enabling the surgeon to position the cutting slot in a desired position.

As will be shown and described herein, the cutting guide may be used to resect portions of a humeral head of a patient's humerus during a shoulder arthroplasty. However, the cutting guide is not so limited and may be used in other surgical procedures to resect or cut other portions of a patient's bone. For example, the cutting guide may be used to facilitate single or multiple cuts in a patient's bone. In some examples, the cutting guide may be used to resect portions of a patient's femur in a hip arthroplasty or a patient's tibial or femur in a knee arthroplasty details of which are provided in International Patent Application No. PCT/US2022/045613, published as WO202305989, entitled “Cutting Guide Systems,” the entirely of which is hereby incorporated by reference. In some examples, the cutting guide may be used to perform one or more cuts in a high tibial osteotomy (HTO) procedure used to correct angular deformities of a patient's knee. As such, the present disclosure should not be limited to any particular surgical procedure or patient's bone unless explicitly claimed.

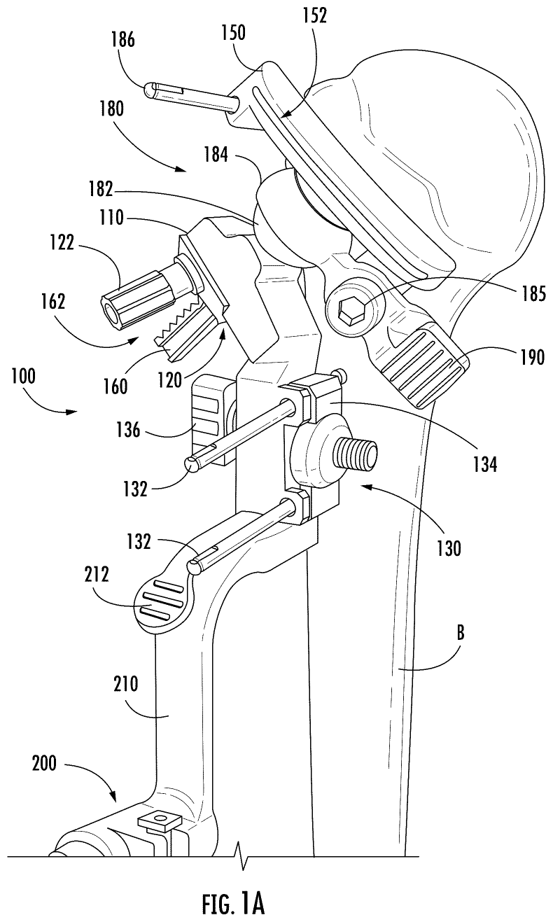

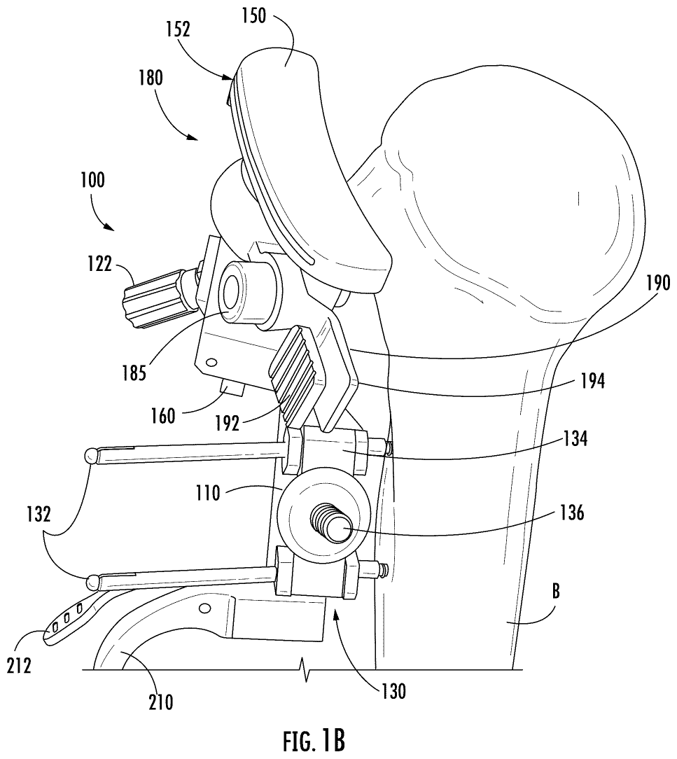

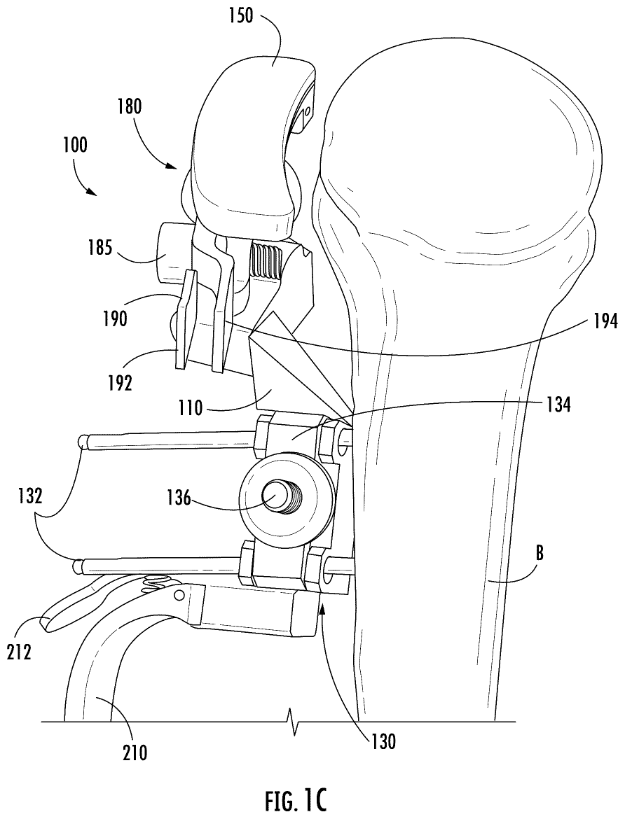

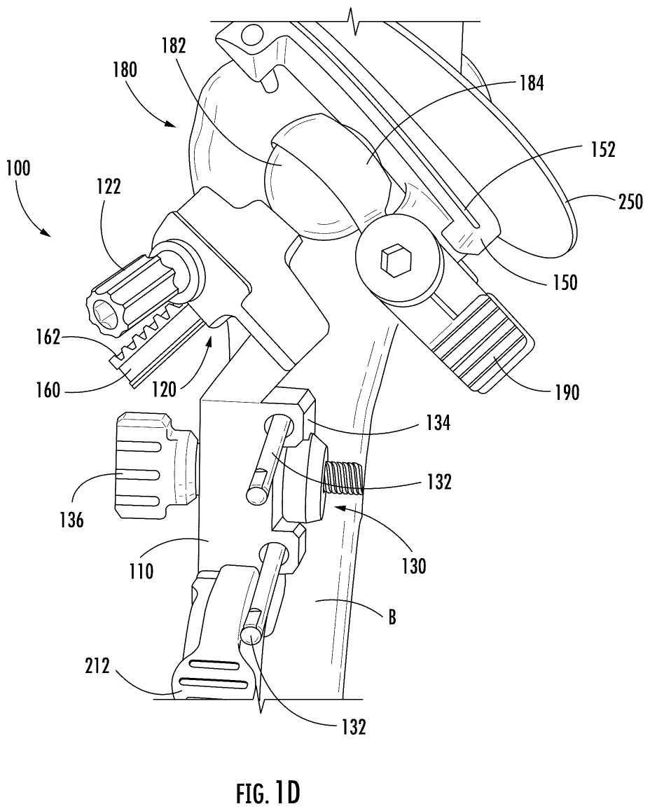

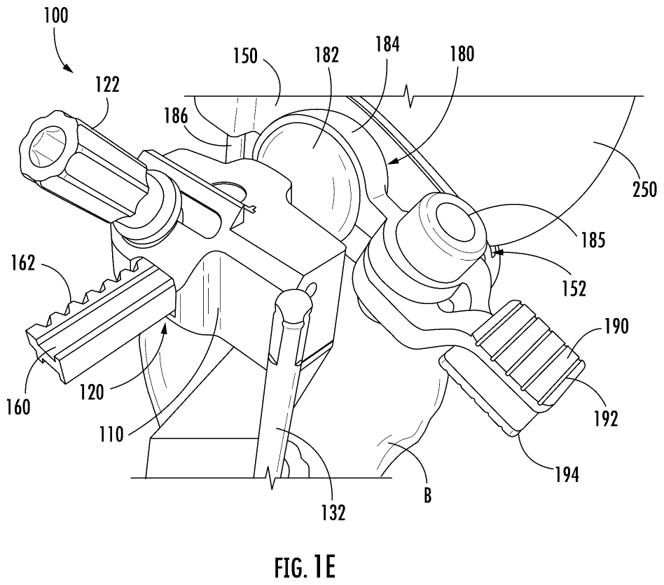

In accordance with one or more features of the present disclosure, with reference to FIGS. 1A-1E, the cutting guide 100 includes a first or main body component 110 (terms first or main used interchangeably herein without the intent to limit or distinguish) and a second or cutting component 150 (terms used interchangeably herein without the intent to limit or distinguish), the cutting component 150 including a cutting slot 152 arranged and configured to receive a cutting instrument or saw (not shown) to resect or cut the patient's bone B (e.g., to resect or cut portions of a humeral head of a patient's humerus as illustrated in the drawings). As such, the cutting component is referred to herein as a cutting slot component 150. In addition, the cutting slot component 150 is coupled to the body component 110 via a ball-joint coupling mechanism 180 (e.g., a ball-and-socket clamping mechanism) for selectively positioning and securing the position of the cutting slot component 150 relative to the body component 110. The ball-joint coupling mechanism 180 including a ball 182 (e.g., a spherical ball or the like) at least partially received within a socket 184.

In addition, in some examples, the cutting guide 100 may further include a shaft 160, the ball 182 of the ball-joint coupling mechanism 180 being located at a first end of the shaft 160, the shaft 160 including a plurality of splines or grooves 162 at a second end thereof. The body component 110 may include an opening 120 for receiving the shaft 160 therein. In addition, the body component 110 may include a knob 122 for interacting with, or engaging, the splines or grooves 162 formed on the shaft 160. Thus arranged, in use, rotation of the knob 122 translates (i.e., moves) the shaft 160, and hence the ball 182 and ball-joint coupling mechanism 180, which moves the cutting slot component 150 relative to the body component 110 along a longitudinal axis of the shaft 160. As such, rotation of the knob 122 adjusts a longitudinal position of the cutting slot component 150, and hence the cutting slot 152, relative to the body component 110.

Alternatively, it should be appreciated that alternate mechanisms may be used to selectively secure the position of the shaft. For example, a ratcheting mechanism or a linkage mechanism may be used.

In addition, as previously mentioned, the cutting slot component 150 and the body component 110 include, or are coupled together via, a ball-joint coupling mechanism 180 (e.g., a ball-and-socket clamping mechanism) for selectively coupling, positioning, and securing the position of the cutting slot component 150 relative to the body component 110. For example, as best illustrated in FIG. 1D, the cutting slot component 150 may be operatively coupled to, or include, the socket 184 and the body component 110 may be operatively coupled to, or include, the ball 182 via, for example, the shaft 160. Thus arranged, movement of the socket 184 relative to the ball 182 moves the cutting slot component 150 relative to the body component 110.

In addition, the cutting guide 100 may include a quick release mechanism. For example, as illustrated, the quick release mechanism may be a push-button release or spring tab mechanism 190 moveable between first and second positions. In the first position, the ball-joint coupling mechanism 180 is locked or secured so that movement of the cutting slot component 150 relative to the body component 110 is prevented, or at least minimized. In the second position, the ball-joint coupling mechanism 180 is released so that the cutting slot component 150 can be moved relative to the body component 110 thereby enabling the surgeon to position the cutting slot 152 in a desired position. That is, with the push-button release or spring tabs 190 in the first position, a clamping or spring force is applied to the ball-joint to prevent movement thereof, and hence prevent, or at least inhibit, movement of the cutting slot component 150 relative to the body component 110. However, with the push-button release or spring tabs 190 in the second position (e.g., with the surgeon pressing or squeezing the first and second arms 192, 194), the clamping or spring force is removed thereby enabling the cutting slot component 150 to move relative to the body component 110. For example, with the clamping or spring force removed, the cutting slot component 150 can move such as, for example, rotate about the ball-joint interface to rotate the cutting slot component 150 relative to the body component 110. In addition, and/or alternatively, in some examples, with the push-button release or spring tabs 190 in the second position, the cutting slot component 150 can be quickly disconnected from and reattached to the body component 110. For example, with the push-button release or spring tabs 190 in the second position (e.g., with the surgeon pressing or squeezing the first and second arms 192, 194), the cutting slot component 150 can be disconnected from and reattached to the body component 110. For example, the socket 184 may be decoupled from the ball 182 thereby disconnecting the cutting slot component 150 from the body component 110. This may be beneficial, for example, during registration of the patient's bone to a computer-assisted surgical system, which typically uses a probe device to touch points/areas on the boney surface. In use, quick removal of the cutting slot component 150 provides increased access to the boney surfaces of the patient's bone (e.g., humerus) while saving the surgeon time and effort during this step. Thereafter, the socket 184 can be reconnected or coupled to the ball 182 thereby connecting the cutting slot component 150 to the body component 110.

In addition, due to spherical configuration of the ball-joint coupling mechanism, the cutting slot component 150 can be freely moved along multiple rotational degree of freedoms allowing for easy simultaneous adjustment of the inclination and version angles of the resection plane (e.g., the ball-joint coupling mechanism allows three (3) rotational degrees of freedom to be adjusted at once).

Alternatively, it should be appreciated that alternate release mechanisms may be used to selectively release and secure the position of the ball-joint coupling mechanism. For example, a button may be used to selectively release and secure the position of the ball-joint coupling mechanism. In use, pressing or moving the button to the second position may release the clamping or spring force on the ball-joint coupling mechanism thereby enabling the position of the cutting slot component to be adjusted relative to the body component.

As best illustrated in FIG. 1C, in use, the push-button release or spring tabs 190 include first and second arms 192, 194. In use, moving (e.g., pressing) a first end of the push-button release or spring tabs 190 causes a second end the first and second arms 192, 194 to move apart from each other, which releases the clamping or spring force on the ball-joint (e.g., pressing the push-button release or spring tab opens the socket joint). In some examples, the push-button release or spring tabs 190 may be biased toward the first position (e.g., the first and second arms are biased apart so that the push-button release or spring tabs are biased toward the first position). In some examples, the socket 184 may be arranged and configured to be slightly undersized relative to the ball 182. Thus arranged, the socket 184 may be stretched when coupled with the ball 182 thereby applying an inherent spring force. Pressing on the push-button release or spring tabs 190 relieves this spring force on the ball 182.

In use, once the position of the cutting slot 152 has been properly positioned in its desired location to facilitate the desired planar cut in the patient's bone B, a locking screw 185 may be tightened and/or inserted into, for example, the release mechanism, to more securely lock the ball-joint coupling mechanism 180 to prevent any unintentional or unwanted movement of the ball-joint, and hence the cutting slot component 150 relative to the body component 110. In addition, and/or alternatively, once the position of the cutting slot 152 has been properly positioned in its desired location, a bone pin 186 may be inserted through an opening formed in the cutting slot component 150 to secure the position of the cutting slot component 150, and hence the cutting slot 152.

In use, the locking screw 185 may have any suitable configuration now known or hereafter developed. For example, as shown, the locking screw 185 may include an internal bore arranged and configured to receive an external hex. Alternatively, the locking screw 185 may include external grooves or knurling to facilitate hand-tightening. In some examples, the locking screw 185 may include an enlarged diameter head portion to indicate and/or further facilitate hand-tightening.

In use, the cutting guide 100 may be coupled to the patient's bone B (e.g., the patient's humerus) by any suitable coupling mechanism now known or hereafter developed. As illustrated, in some examples, the cutting guide 100 may include a clamping mechanism 130 for receiving one or more bone pins 132 (first and second bone pins illustrated in the FIGS., although more or less bone pins may be used). That is, in some examples, the cutting guide 100 may include a clamp plate 134 and a threaded knob 136 (e.g., a knob with an externally threaded shaft extending therefrom, or associated therewith). As will be readily appreciated by one of ordinary skill in the art, in use, rotation of the threaded knob 136 moves the clamp plate 134 relative to the body component 110. Thus arranged, in use, a surgeon can insert one or more bone pins 132 into the patient's bone B. Thereafter, the cutting guide 100, and more specifically, the body component 110, may be coupled to the bone pins 132, and the threaded knob 136 rotated to secure the bone pins 132 in-between the clamp plate 134 and the body component 110 thereby coupling the cutting guide 100, and more specifically, the body component 110, to the patient's bone B, although this is but one configuration and other coupling mechanisms for coupling the cutting guide 100 to the patient's bone B are envisioned including, for example, a bone clamp, fasteners, etc. It should be noted that given the additional flexibility provided by the cutting guide 100 in accordance with one or more features of the present disclosure, precise placement of the bone pins 132 is reduced.

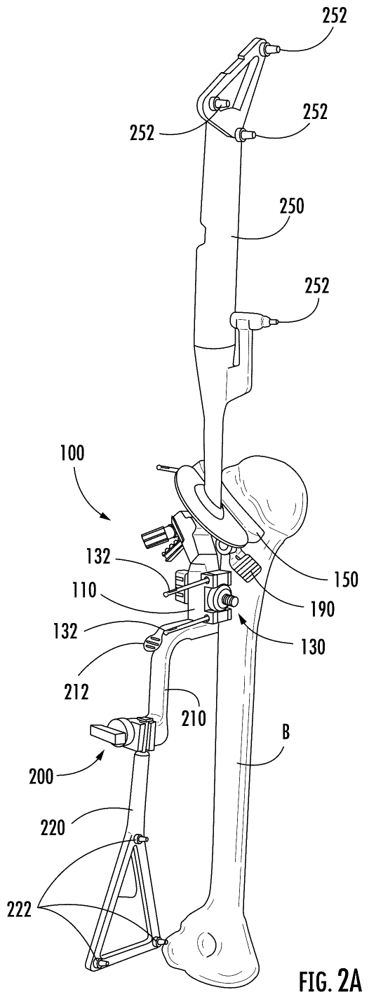

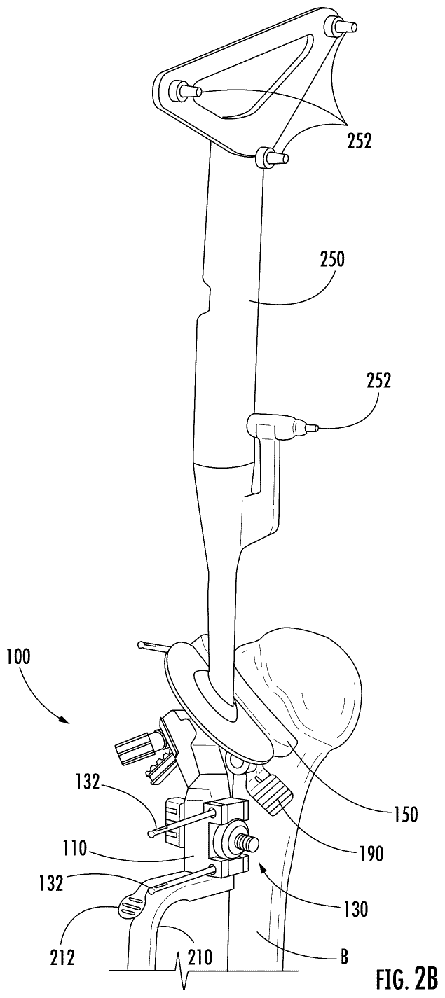

With additional reference to FIGS. 2A and 2B, with the cutting guide 100 secured to the patient's bone B and with the cutting slot 152 positioned in its desired position, a plane visualizer 250 may be inserted into the cutting slot 152 of the cutting guide 100. As illustrated, in some examples, the plane visualizer 250 may include a plurality of fiducial markers 252. Thus arranged, during a computer-assisted surgical procedure, the cutting plane (i.e., the resection plane or planar cut) can be precisely tracked and positioned as desired by the surgeon. Additional information on an example of a plane visualizer can be found in U.S. Pat. No. 6,514,259, entitled “Probe and Associated System and Method for Facilitating Planar Osteotomy During Arthroplasty.”

In addition, as illustrated, the cutting guide 100 may include, or be coupled to, a tracking array 220 including a plurality of fiducial markers 222. In use, the tracking array 220 may be coupled to the cutting guide 100 by any suitable mechanism now known or hereafter developed. For example, as illustrated, the cutting guide 100 may include a clamping mechanism 200 for coupling to the tracking array 220. In use, the tracking array 220 provides precise location information to the computer in a computer-assisted surgical system to precisely identify and/or locate the position of the patient's bone B (e.g., the patient's humerus). That is, the tracking array 220 enables registration in space of the precise location of the patient's bone B.

Thus arranged, with the tracking array 220 providing precise information for locating the patient's bone B and with the plane visualizer 250 providing precise information on the location or trajectory of the cutting slot 152, and hence the cutting plane (i.e., the resection plane or planar cut) for resecting the patient's bone B, the surgeon is able to quickly and precisely (i.e., accurately) position and fine tune the location of the cutting slot 152 and hence the location of the resection plane, by adjusting the knob 122 to translate the shaft 160 and/or by adjusting the position of the cutting slot 152 by adjusting the relative position between the cutting slot component 150 relative to the body component 110, which is affixed to the patient's bone B.

In addition, as illustrated, the cutting guide 100 may include an arm or segment 210 and a secondary or supplemental release mechanism 212. In some examples, the segment 210 acts as an intermediary arm extending between the clamping mechanism 200 for coupling the tracking array 220 and the body component 110 of the cutting guide 100. As illustrated, in some examples, the secondary or supplemental release mechanism 212 may be configured as a quick release mechanism such as, for example, a push-button or release tab moveable between first and second positions. In the first position, the segment 210 is locked or coupled to the body component 110 of the cutting guide 100. In the second position, the segment 210 is released so that the segment 210 and tracking array 220 can be decoupled from the body component 110 of the cutting guide 100. Thus arranged, the secondary or supplemental release mechanism 212 is arranged and configured to release the segment 210 from the body component 110 so that, during use, the end user can depress the release mechanism 212 to decouple or release the segment 210 and the tracking array 220 coupled thereto as a single unit. As such, the cutting guide 100 includes a quick-connect mechanism for removing the segment 210 and tracking array 220. In use, such arrangement facilitates, removal and reattachment of the tracking array 220 while maintaining the ability to track the patient's bone (e.g., a precise interface is created that allows the surgeon to remove and reattach the interface while maintaining accuracy of the tracking).

Thus arranged, in accordance with one or more features of the present disclosure, a cutting guide including a cutting slot and a geared shaft including a spherical ball on an end thereof is provided. The cutting slot being coupled to a spherical ball at the end of the geared shaft to enable actuation or translation in a single direction. In use, a ball-joint coupling mechanism (e.g., a socket-like clamping mechanism) and quick release tabs enable selective movement (e.g., rotation) of the cutting slot relative to the geared shaft to provide multiple degrees of rotational freedom to the surgeon in positioning the cutting slot. As a result, the cutting slot can be actuated or move linearly along the direction of the geared shaft and freely rotated about the spherical ball-socket interface. In use, with the release tabs in their normal, at rest position, a spring force in the clamping mechanism holds the socket/clamp in position relative to the spherical ball. When the release tabs are pressed (e.g., squeezed together) the spring tension on the spherical ball is relieved, and the position of the cutting slot can be adjusted freely, or completely removed from the spherical ball. When the desired position of the cutting slot has been achieved, the ball-joint coupling mechanism (e.g., a socket-like clamping mechanism) can be tightened using a locking screw.

In some examples, the cutting guide may be manufactured from any suitable material now known or hereafter developed including, for example, a metal, a polymer, a composite, or the like. In some examples, the cutting guide including the ball-joint coupling mechanism (i.e., the spherical ball and socket) may be manufactured from a metal such as, for example, a stainless steel such as, for example, 17-4 stainless steel, or other suitable metals such as, for example, aluminum.

In some examples, the surface of the ball and socket may be smooth. Alternatively, in some examples, the surface of the ball and socket may include a textured surface. For example, the outer surface of the ball and/or the inner surface of the socket may include a blasted, texturized surface. In yet other examples, the outer surface of the ball may include a plurality of dimples and the inner surface of the socket may include a plurality of nubs, or vice-versa, for interacting with the plurality of dimples that compress the ball to provide increased effective fixation.

In some examples, the socket may be slightly undersize relative to the ball to provide a natural compressive fit to help facilitate maintaining the position of the cutting slot when the push-button release or spring tabs are not depressed.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. A cutting guide for resecting a portion of a patient's bone, the cutting guide comprising:
a body component;
a cutting slot component including a cutting slot arranged and configured to receive a cutting instrument or saw for resecting the portion of the patient's bone;
a ball-joint coupling mechanism for coupling the cutting slot component to the body component, the ball-joint coupling mechanism including a ball and a socket; and
a release mechanism moveable between a first position and a second position, wherein, in the first position, the release mechanism applies a clamping or spring force on the ball-joint coupling mechanism to inhibit movement of the cutting slot component relative to the body component, and in the second position, the release mechanism releases the clamping or spring force on the ball-joint mechanism so that the cutting slot component can be rotated relative to the body component.

2. The cutting guide of claim 1, wherein the release mechanism comprises a push-button tab including first and second arms, wherein pressing the first and second arms towards each other causes the release mechanism to move from the first position to the second position.

3. The cutting guide of claim 2, wherein pressing the first and second arms toward each other causes an opposite end of the first and second arms to move away from each other causing the clamping or spring force on the ball-joint coupling mechanism to be reduced.

4. The cutting guide of claim 2, wherein the first and second arms are biased apart so that the release mechanism is biased toward the first position.

5. The cutting guide of claim 1, wherein the cutting slot component further comprises a locking screw wherein tightening the locking screw further secures a relative position of the cutting slot component relative to the body component.

6. The cutting guide of claim 5, wherein the locking screw includes an enlarged head portion with a knurled outer surface to facilitate hand-tightening of the locking screw.

7. The cutting guide of claim 1, wherein the cutting slot component further comprises an opening for receiving a bone pin for further securing the position of the cutting slot component relative to the body component.

8. The cutting guide of claim 1, wherein the cutting device further includes a shaft, the ball being located at a first end of the shaft, the shaft including a plurality of splines at a second end thereof.

9. The cutting guide of claim 8, wherein the body component includes an opening for receiving the shaft therein and a knob for engaging the plurality of splines formed on the shaft so that, in use, rotation of the knob translates the shaft, and hence the cutting slot component relative to the body component along a longitudinal axis of the shaft.

10. The cutting guide of claim 1, wherein in the second position, the ball-joint coupling mechanism enables multiple rotational degree of freedoms to enable a surgeon to position the cutting slot component relative to the body component.

11. The cutting guide of claim 10, wherein the ball-joint coupling mechanism enables simultaneous adjustment of an inclination angle and a version angle of a resection plane.

12. The cutting guide of claim 1, wherein the body component is coupled to the patient's bone via a coupling mechanism.

13. The cutting guide of claim 12, wherein the coupling mechanism comprises a clamping mechanism arranged and configured to receive one or more bone pins.

14. The cutting guide of claim 13, wherein the clamping mechanism includes a clamp plate and a threaded knob, in use, rotation of the threaded knob moves the clamp plate relative to the body component to secure the body component to the one or more bone pins.

15. The cutting guide of claim 1, further comprising a plane visualizer arranged and configured to be at least partially inserted into the cutting slot, the plane visualizer including a plurality of fiducial markers to assist in locating and tracking a position of the cutting slot.

16. The cutting guide of claim 15, wherein further comprising a tracking array including a plurality of fiducial markers to assist in locating a position of the patient's bone.

17. The cutting guide of claim 16, further comprising a secondary release mechanism moveable between first and second positions to facilitate removal of the tracking array while maintaining accurate tracking of the patient's bone.

18. The cutting guide of claim 1, wherein an outer surface of the ball and an inner surface of the socket comprise a textured surface.
